SEQUENCE LISTING (2 sequences)

SEQ ID NO: 1 - DNA, 39 bp, Artificial Sequence
  primer for amplification
gtagagctccaccatgacttcccattatgtgattgccat

SEQ ID NO: 2 - DNA, 36 bp, Artificial Sequence
  primer for amplification
gtgtctagactagtccagaccatggaaaattggtgg

(19) United States
(12) Patent Application Publication
(10) Pub. No.: US 2009/0263389 A1
(43) Pub. Date: Oct. 22, 2009

(54) Use of TGF-Beta Antagonists to Treat or to Prevent Chronic Transplant Rejection

(75) Inventors: Shaf Keshavjee, Toronto (CA); Judith A. St. George, Hudson, MA (US); Mingyao Liu, Richmond Hill (CA)

Correspondence Address: GENZYME CORPORATION;LEGAL DEPARTMENT, 15 PLEASANT ST CONNECTOR, FRAMINGHAM, MA, 01701-9322, US

(73) Assignees: Genzyme Corporation, Cambridge, MA (US); University Health Network, Toronto (CA)

(21) Appl. No.: 12/469,554

(22) Filed: May 20, 2009

Related U.S. Application Data
(63) Continuation of application No. 10/348,665, filed on Jan. 21, 2003.
(60) Provisional application No. 60/350,529, filed on Jan. 22, 2002.

(51) Int. Cl. A61K 39/395; A61K 38/17; A61K 31/711

(52) U.S. Cl. 424/133.1; 514/8; 514/44 R

(57) ABSTRACT

Effective use of a TGF-β antagonist to treat or to prevent loss of transplant function is described herein. Use of a TGF-β antagonist is demonstrated to effectively prevent loss of organ function in a host due to chronic rejection in which TGF-β-mediated fibroproliferation is a characteristic. Expression in situ of a TGF-β antagonist in the form of a recombinant receptor, i.e., TGF-β type III receptor (TGFBIIIR) showed prevention of bronchiolitis obliterans in comparison to untreated controls in a rat lung transplant model. This provides an effective method for preventing or inhibiting chronic rejection of transplant organs such as lung, kidney, liver and heart in vertebrate hosts including human hosts.

### CROSS REFERENCE TO RELATED APPLICATIONS

This application claims priority under 35 U.S.C. § 119(e) to U.S. provisional application Ser. No. 60/350,529, filed Jan. 22, 2002.

### FIELD OF THE INVENTION

The present invention is in the fields of molecular biology and organ transplantation. The present invention is directed to novel methods for treating or preventing rejection of transplanted organs or tissues by the use of an effective inhibitor of TGF-β.

### BACKGROUND OF THE INVENTION

Organ transplantation has become an important therapy for patients facing loss of organ function due to disease or injury. In the United States, for example, for the period from January 1997 through December 1998 (i.e., the most recent period with complete 3-year follow-up statistics), more than 1600 lung transplants, more than 4000 heart transplants, more than 7000 liver transplants, more than 400 pancreas transplants, and more than 22,000 kidney transplants were performed.

Allotransplantation of heart, lung, kidney, pancreas and liver in human hosts is common, and xenotransplantation, especially of porcine or simian organs into humans, has also shown some promising results. Whereas one-month success rates for all types of allotransplants are greater than 90%, the 1-year survival rate of transplants is significantly lower and the 3-year survival rate for all organs other than kidney is 74% or lower (79% survival rate for kidney transplant). See, United Network for Organ Sharing (UNOS), 2001 statistics.

The success of organ transplants depends on avoiding rejection of the transplant. The forms of transplant rejection are clinically classified by their timeframes and histologies. Hyperacute rejection occurs in minutes to hours following transplant, acute rejection typically occurs within 1-30 days, and chronic rejection occurs thereafter, sometimes taking several months to years. Hyperacute and acute rejection are largely understood to be the result of immunological attack on the donor organ, prompted by the lack, to varying degrees, of histocompatibility between the donor organ and the host. Immune suppression is sometimes successful in overcoming acute rejection, and the use of immunosuppressive agents such as cyclosporin A is nearly universal in transplant recipients.

Whereas hyperacute and acute rejection are suppressed with immunosuppressive protocols, treatment for chronic rejection is less well defined. It is understood that acute rejection and chronic rejection have significantly different characteristics as immune responses. Chronic rejection occurs over time and is usually the result of a prolonged process of wound healing the host undergoes post-transplant. It involves multiple factors and processes of the host, and is this sometimes difficult to detect and treat in a time frame that will save the transplant.

For most organs, the most definitive way of showing that rejection is occurring is by biopsy of that organ. For practical reasons, however, biopsies are not always done and are particularly less practical when chronic rejection is suspected. Chronic rejection of a transplant organ is generally characterized as failure of the organ after it has begun to perform its function in the recipient or host. Thus, chronic rejection is commonly monitored by a decrease in organ function which, if unarrested, results in failure of the organ, infection, and necrosis of organ tissue. Chronic rejection is identified, commonly too late for treatment that can save the transplant, by pathogenic fibrosis, which is characterized by extensive deposition of the extracellular matrix proteins: collagen, fibronectin, and elastin, and by emergence of cells with the myofibroblast phenotype. Fibrosis becomes a telltale characteristic of chronic rejection where fibrogenesis is observed to damage organ microstructures or to block passages that need to remain open for organ function.

Recent studies have explored the mechanisms of chronic rejection with an emphasis on the existence of several interrelated inflammatory cascades and the complexity of this immune response. It has been found that the processes involved in wound healing and chronic rejection are complex, and many positive correlations with chronic rejection exist. For example, regulatory and cytotoxic molecules such as TNF-α, TGF-β, IL-10, IL-2, and granzyme B are all upregulated in rejection episodes (see, Suthanthiran M., 1997, F. Kidney Int. Suppl., 58:S15-21); and cyclosporin A, which is ubiquitous in human transplant operations, has also been implicated in fibrosis (see, e.g., Cuhaci et al., 1999, Transplantation, 68(6):785-90). As a result, a single therapy for the prophylaxis or treatment of chronic rejection has been precluded partly due to the complexity of the cellular mechanisms involved in chronic rejection.

While significant improvements have been made in the treatment and prevention of hyperacute and acute transplant rejection, most grafts or transplants will ultimately yield to chronic rejection. This reflects the extent of our knowledge of the mechanisms that drive these processes. As yet, no current treatment protocol or drug has been proven to effectively control chronic rejection or regulate the immunoregulatory factors which determine chronic rejection. Accordingly, there is a need in the art to determine whether inhibition or control of a single factor or small number of factors in chronic rejection would have an impact on chronic rejection of transplants.

### SUMMARY OF THE INVENTION

We have noted that fibrosis is a common factor in chronic rejection of all types of organ transplants, and that TGF-β is often elevated (along with many other factors) in chronic rejection due to fibrosis. For example, in lung transplant, fibroproliferation correlated with upregulation of TGF-β following transplant leads to a fibrous destruction of small airways known as bronchiolitis obliterans (see, e.g., Charpin et al., 1998, Transplantation, 65(5):752-755; El-Gamel et al., 1998, Eur. J. Cardiothorac. Surg., 13(4):424-30); in renal transplant, fibrosis of the tubulointerstitium leading to decline in renal transplant function was correlated with increased expression of TGF-β and secretion of TGF-β in urine (see, e.g., Cuhaci et al., 1999, Transplantation, 68(6):785-790; Boratynska, 1999, Ann. Transplant., 4(2):23-28); in heart transplant, total TGF-β and endogenous TGF-β expression (mRNA) was sometimes correlated with accelerated atherosclerosis (see, Little et al., 1999, Transpl. Int., 12(6):393-401); and in liver regeneration, TGF-β is believed to be an inhibitor of hepatocyte proliferation and to induce fibrosis in chronic liver disease (see, e.g., Fausto et al., 1991, Ciba Found. Symp., 157:165-174; discussion 174-7.

TGF-β is a member of a superfamily of proteins that control development and tissue. homeostasis in organisms as diverse as drosophila and humans (Grande, 1997, Proc. Soc. Exp. Biol. Med., 214(1):27-40). TGF-β functions in a variety of biological processes including energy production in mitochondria, regulation of vascular tone, cellular differentiation, proliferation, and apoptosis. TGF-β is best known as a cytokine responsible for activating extracellular matrix production associated with wound healing. The effects of TGF-β on cell proliferation are complex and as yet little is known about the mechanisms which induce activation of TGF-β or elicit wound healing and tissue regeneration.

We have now discovered that inhibition of TGF-β alone, i.e., by introduction of a TGF-β antagonist, is useful for inhibiting chronic rejection of transplant organs. This represents the first demonstration that chronic rejection associated with fibroproliferation can be prevented using a direct inhibitor of TGF-β function.

Although TGF-β is best known as a cytokine responsible for activating extracellular matrix production associated with wound repair, and although it remains the premier fibrogenic cytokine of study concerning fibrosis in particular, TGF-β displays ubiquitous and diverse biologic functions. The present invention teaches that TGF-β plays a significant role in chronic rejection of transplanted organs including lung, kidney, heart, pancreas, and liver transplants, and demonstrates that TGF-β antagonists act as effective therapeutics, preventing loss of transplant function.

It is therefore an object of the present invention to provide a method for treating or preventing chronic rejection of a transplant organ comprising administering to an individual susceptible to or showing symptoms of chronic rejection a pharmaceutically effective amount of a TGF-β antagonist.

In a related aspect, the present invention provides for the use of a TGF-β antagonist in the preparation of a pharmaceutical composition for treating a transplant recipient to prevent or delay rejection of the transplant.

The present invention further relates to the use of a TGF-β antagonist to maintain transplant function in a host (recipient) mammal, or to slow, to halt, to prevent, or to reverse loss of transplant function. Preferred embodiments of the present invention include administering a pharmaceutically effective amount of a TGF-β antagonist to maintain and to regulate desirable levels of transplant organ function or to reduce or inhibit fibrosis in the transplant.

TGF-β antagonists of the present invention include any molecule that is able to decrease the amount or activity of TGF-β, either within a transplant organ or within a transplant recipient. TGF-β antagonists of the present invention also include any nucleic acid sequence that encodes a molecule capable of decreasing the amount or activity of TGF-β. Preferably, TGF-β antagonists include: antibodies directed against one or more isoforms of TGF-β; TGF-β receptors and soluble fragments thereof that bind to TGF-β; antibodies directed against TGF-β receptors; latency associated peptide; large latent TGF-β; TGF-β inhibiting proteoglycans such as fetuin, decorin, biglycan, fibromodulin, lumican and endoglin; somatostatin; mannose-6-phosphate; mannose-1-phosphate; prolactin; insulin-like growth factor II; IP-10; the tripeptide arg-gly-asp and peptides containing the tripeptide; TGF-β inhibitory extracts from plants, fungi, or bacteria; antisense oligonucleotides, e.g., that inhibit TGF-β gene transcription or translation; proteins involved in TGF-β signaling, including SMADs, MADs, Ski, Sno; and any mutants, fragments or derivatives of the above-identified molecules that retain the ability to inhibit the activity of TGF-β. More preferably the TGF-β antagonist is a human or humanized monoclonal antibody that blocks TGF-β binding to its receptor (or fragments thereof such as F(ab)₂ fragments, Fv fragments, single chain antibodies and other forms or fragments of antibodies that retain the ability to bind to TGF-β. A preferred monoclonal antibody is a human or humanized form of the murine monoclonal antibody obtained from hybridoma 1D11.16 (ATCC Accession No. HB 9849). Another preferred inhibitor of TGF-β function is a soluble TGF-β receptor, especially TGF-β type II receptor (TGFBIIR) or TGF-β type III receptor (TGFBIIIR, or betaglycan) comprising, e.g., the extracellular domain of TGFBIIR or TGFBIIIR, most preferably a recombinant soluble TGF-β receptor (rsTGFBIIR or rsTGFBIIIR). Polypeptide inhibitors such as the soluble TGF-β receptors may be effectively introduced via gene transfer, as demonstrated herein.

### BRIEF DESCRIPTION OF THE DRAWINGS

FIG. 1 is a schematic diagram of recombinant adenovirus vector Ad2 TGFBIIIR E3 Δ2.9, for in vivo expression of rsTGFBIIIR. The vector is prepared by insertion of an expression cassette for human rsTGFBIIIR in place of the E1 region at the ICEU I site in pADQUICK (formerly called pAd_{vantage}; see, Souza et.al., 1999, Biotechniques, 26:502-8)

FIG. 2 is a bar graph illustrating time-dependent inhibitory effect of adenoviral-vectored recombinant soluble TGF-β type III Receptor (rsTGFBIIIR) cDNA on development of lumenal obliteration in rat tracheal allografts. The graph shows effect of adenovirus containing rsTGFBIIIR injected at the site of transplants on day 0 (D0), day 5 (D5), and day 10 (D10), compared to untreated control (CO). TGFBIIIR expression vector administered on day 5 inhibited development of fibrous airway obliteration (p=0.06 vs. CO).

FIG. 3 is a bar graph illustrating inhibitory effect of local (topical) administration of adenoviral-mediated soluble TGFBIIIR gene transfer on development of lumenal obliteration in rat tracheal allografts.

FIG. 4 shows representative histology sections from tracheal allografts treated or untreated with TGF-β antagonist. Adenoviruses containing soluble TGFBIIIR cDNA, or an empty vector, were injected at the site of transplants (topical) or intramuscularly on Day 5 after transplant of the lung tissue. Representative histology sections of allografts on Day 21 are presented: CO=untreated control; TG=topical gene; TV=topical empty vector; IMG=intramuscular gene. Injection at the transplant site (topical administration) of soluble TGFBIIIR vector preserved lumenal patency, whereas the sections from other groups showed obstruction of lumenal sections with fibrosis (Cf. TG with CO, TV, IMG).

### DETAILED DESCRIPTION OF THE INVENTION

Disclosed herein is the first report describing the effective use of TGF-β antagonists to block chronic destruction of transplant organs due to TGF-β-mediated fibroproliferation. As demonstrated herein, TGF-β antagonists are useful to prevent and to reduce loss of transplant function. It is demonstrated for the first time herein that chronic rejection of transplanted organs can be arrested by administration of a TGF-β antagonist that directly inhibits TGF-β activity. The present invention thus provides a means for preventing or delaying chronic rejection of transplanted organs, particularly lung, heart, kidney, pancreas, and liver allografts. The present invention provides a method for arresting destructive fibrosis in transplant tissue, which is a key indicator of chronic rejection but an indicator usually detected when rejection or failure of the transplant is inevitable.

The present invention is directed to a method for treating or delaying transplant rejection associated with fibrosis comprising administering to an individual receiving the transplant a pharmaceutically effective amount of a TGF-β antagonist. The present invention is also directed to use of a TGF-β antagonist for preparation of a pharmaceutical composition useful for treating or delaying chronic rejection of a transplant organ. In particular embodiments, the present methods and compositions are useful to treat or delay chronic rejection of a transplanted lung, heart, pancreas, kidney, or liver, or any other transplantable organ or tissue susceptable to fibrosis or TGF-β-mediated chronic rejection.

Chronic rejection can result from a range of specific disorders characteristic of the particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis, in kidney transplants, such disorders include, obstructive nephropathy, nephrosclerosis, tubulointerstitial nephropathy; in liver transplants, such disorders include disappearing bile duct syndrome.

As used herein, the term “transplant” when used as a noun refers to a whole organ (such as lung, kidney, heart, liver) from a donor individual or a functional part of an organ (such as a lobe of a donor liver, a heart valve, a section of artery or vein, skin graft) which is excised from the donor for transplantation in a recipient individual (host). “Transplant” may also refer to any grafts, tissues or cells that are foreign to a recipient and, for the purposes of the invention, are susceptible to failure from chronic rejection. An allotransplant is a transplant excised from a donor that is the same species as the recipient; a xenotransplant is a transplant excised from a donor that is of a different species than the recipient.

As used herein, reference to “treating or delaying chronic rejection of a transplant” generally refers to any process that functions to slow, to halt (including stopping initial onset), or to reverse loss of transplant function. Such treatment may be administered prior to display of a symptom of chronic rejection or after onset of such a symptom.

“Loss of transplant function”, as used herein, refers to any physiological disruption or dysfunction of the normal processes the organ or tissue exhibits in the donor animal. For the purposes of this invention, mere physical abnormalities (including fibrosis) of the transplanted organ are not considered, per se, organ dysfunctions, or a disease or disorder of the organ. Loss of transplant function specifically refers to the diminution in the processes that the organ normally performs in the donor. For example, in a kidney transplant, diminution of pressure filtration, selective reabsorption, or tubular secretion indicate loss of kidney function, as do medullary hypoperfusion; medullary hypoxia, including hypoxic tubular injury, tubular necrosis, formation of protein casts and tubular obstruction, or other manifestations that reduce tubular flow; as well as manifestations that reduce medullary blood flow such as ischemia and other vasa recta injury.

As used herein, the term “recombinant” is used to describe non-naturally altered or manipulated nucleic acids, host cells transfected with exogenous (non-native) nucleic acids, or polypeptides expressed non-naturally, through manipulation of isolated DNA and transformation of host cells. Recombinant is a term that specifically encompasses DNA molecules which have been constructed in vitro using genetic engineering techniques, and use of the term “recombinant” as an adjective to describe a molecule, construct, vector, cell, polypeptide or polynucleotide specifically excludes naturally occurring such molecules, constructs, vectors, cells, polypeptides or polynucleotides. “Recombinant expression” of a protein includes not only expression of a man-made, recombinant gene in a host cell but also in situ activation by recombinant means of naturally occurring gene sequences.

As used herein, a “pharmaceutical composition” refers to any composition that contains a pharmaceutically effective amount of one or more active ingredients (e.g., a TGF-β antagonist) in combination with one or more pharmaceutical carriers and/or additives. Determination of suitable pharmaceutical carriers and/or additives useful for a pharmaceutical composition, as well as the form, formulation, and dosage of such composition, is well within the ability of those skilled in the art (see, for example, Remington's Pharmaceutical Sciences, Mack Publishing Co.). Carriers and/or additives may include but are not limited to: excipients; disintegrators; binders; thickeners, lubricants; aqueous vehicles; oily vehicles; dispersants; preservatives; and isotonizing, buffering, solubilizing, soothing and/or stabilizing agents. The proportion of active ingredient(s) in a pharmaceutical composition of the present invention can be appropriately determined by a person of skill in the art based upon, e.g., the transplant host, the transplant host's age and body weight, the transplant host's clinical status, administration time, dosage form, method of administration, and combination of active components, among other factors. Preferably, the pharmaceutical composition of the present invention is low in toxicity and can safely be used in vertebrates, more preferably mammals, and most preferably humans.

As used herein, a “pharmaceutically effective amount” is an amount effective to achieve the desired physiological result in a subject. Specifically, a pharmaceutically effective amount of a TGF-β antagonist is an amount sufficient to decrease the quantity or activity of TGF-β for a period of time sufficient to ameliorate one or more of the pathological processes associated with loss of transplant function. The effective amount may vary depending on the specific TGF-β antagonist selected, and is also dependent on a variety of factors and conditions related to the subject to be treated and the severity of the disorder (for example, the age, weight and health of the patient as well as dose response curves and toxicity data). The determination of a pharmaceutically effective amount for a given agent is well within the ability of those skilled in the art.

“Administration” to a transplant host is not limited to any particular delivery system and may include, without limitation, parenteral (including subcutaneous, intravenous, intramedullary, intraarticular, intramuscular, or intraperitoneal injection) rectal, topical, transdermal or oral (for example, in capsules, suspensions or tablets). Administration to a host may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.). Administration of a TGF-β antagonist to a host individual may also be by means of gene transfer, wherein a nucleic acid sequence encoding the antagonist is administered to the patient (host) in vivo or to cells in vitro, which are then introduced into the patient, and the antagonist is thereafter produced by in situ expression of the product encoded by the nucleic acid sequence. Methods for gene therapy to deliver TGF-β antagonists are also well known to those of skill in the art (see, for example, WO 96/25178; see, also, Examples 1-5, below).

As used herein, “host” refers to any vertebrate recipient of an organ or tissue transplanted from a donor vertebrate. The donor and host may be of the same or different species. The terms “host” and “recipient” are used herein interchangeably.

As used herein, “TGF-β” refers to all isoforms of TGF-β. There are currently 5 known isoforms of TGF-β (1-5), all of which are homologous (60-80% identity) and all of which form homodimers of about 25 kD, and act upon common TGF-β cellular receptors (Types I, II, and III). The genetic and molecular biology of TGF-β is well known in the art (see, for example, Roberts, 1998, Miner. Electrolyte and Metab., 24(2-3):111-119; Wrana, 1998, Miner. Electrolyte and Metab., 24(2-3):120-130.)

As used herein, a “TGF-β antagonist” is any molecule that is able to decrease the amount or activity of TGF-β, either within a cell or within a physiological system. Preferably, the TGF-β antagonist acts to decrease the amount or activity of a TGF-β1, 2, or 3. For example, a TGF-β antagonist may be a molecule that inhibits expression of TGF-β at the level of transcription, translation, processing, or transport; it may affect the stability of TGF-β or conversion of the precursor molecule to the active, mature form; it may affect the ability of TGF-β to bind to one or more cellular receptors (e.g., Type I, II or III); or it may interfere with TGF-β signaling.

A variety of TGF-β antagonists and methods for their production are known in the art and many more are currently under development (see for example, Dennis et al., U.S. Pat. No. 5,821,227). The specific TGF-β antagonist employed is not a limiting feature; any effective TGF-β antagonist as defined herein may be useful in the methods and compositions of this invention. Preferably, the TGF-β antagonist is a TGF-β1, TGF-β2, or TGF-β3 antagonist. Most preferably the antagonist is a TGF-β1 antagonist.

Examples of TGF-β antagonists include, but are not limited to: monoclonal and polyclonal antibodies directed against one or more isoforms of TGF-β (Dasch et al., U.S. Pat. No. 5,571,714; see, also, WO 97/13844 and WO 00/66631); TGF-β receptors, soluble forms of such receptors (preferably soluble TGF-β type III receptor), or antibodies directed against TGF-β receptors (Segarini et al., U.S. Pat. No. 5,693,607; Lin et al., U.S. Pat. No. 6,001,969, U.S. Pat. No. 6,010,872, U.S. Pat. No. 6,086,867, U.S. Pat. No. 6,201,108; WO 98/48024; WO 95/10610; WO 93/09228; WO 92/00330); latency associated peptide (WO 91/08291); large latent TGF-β (WO 94/09812); fetuin (U.S. Pat. No. 5,821,227); decorin and other proteoglycans such as biglycan, fibromodulin, lumican and endoglin (WO 91/10727; Ruoslahti et al., U.S. Pat. No. 5,654,270, U.S. Pat. No. 5,705,609, U.S. Pat. No. 5,726,149; Border, U.S. Pat. No. 5,824,655; WO 91/04748; Letarte et al., U.S. Pat. No. 5,830,847, U.S. Pat. No. 6,015,693; WO 91/10727; WO 93/09800; and WO 94/10187); somatostatin (WO 98/08529); mannose-6-phosphate or mannose-1-phosphate (Ferguson, U.S. Pat. No. 5,520,926); prolactin (WO 97/40848); insulin-like growth factor II (WO 98/17304); IP-10 (WO 97/00691); arg-gly-asp containing peptides (Pfeffer, U.S. Pat. No. 5,958,411; WO 93/10808); extracts of plants, fungi and bacteria (EP-A-813 875; JP 8119984; and Matsunaga et al., U.S. Pat. No. 5,693,610); antisense oligonucleotides (Chung, U.S. Pat. No. 5,683,988; Fakhrai et al., U.S. Pat. No. 5,772,995; Dzau, U.S. Pat. No. 5,821,234, U.S. Pat. No. 5,869,462; and WO 94/25588); proteins involved in TGF-β signaling, including SMADs and MADs (EP-A-874 046; WO 97/31020; WO 97/38729; WO 98/03663; WO 98/07735; WO 98/07849; WO 98/45467; WO 98/53068; WO 98/55512; WO 98/56913; WO 98/53830; WO 99/50296; Falb, U.S. Pat. No. 5,834,248; Falb et al., U.S. Pat. No. 5,807,708; and Gimeno et al., U.S. Pat. No. 5,948,639), Ski and Sno (Vogel, 1999, Science, 286:665; and Stroschein et al., 1999, Science, 286:771-774); and any mutants, fragments or derivatives of the above-identified molecules that retain the ability to inhibit the activity of TGF-β.

In a preferred embodiment, the TGF-β antagonist is a human or humanized monoclonal antibody that blocks TGF-β binding to its receptor, or fragments thereof such as F(ab)₂ fragments, Fv fragments, single chain antibodies and other forms of “antibodies” that retain the ability to bind to TGF-β. In one embodiment, the TGF-β antagonist is a human antibody produced by phage display (WO 00/66631). In a more preferred embodiment, the monoclonal antibody is a human or humanized form of the murine monoclonal antibody obtained from hybridoma 1D11.16 (ATCC Accession No. HB 9849, described in Dasch et al., U.S. Pat. No. 5,783,185).

An additional preferred embodiment of the present invention involves the use of a vector suitable for expression of a TGF-β receptor or binding partner, preferably a soluble receptor or binding partner. More preferably, administration of a soluble TGF-β antagonist is effected by gene transfer using a vector comprising cDNA encoding the soluble antagonist, most preferably cDNA encoding the extracellular domain of TGF-β type II (rsTGFBIIR) or type III receptor (rsTGFBIIIR), which vector is administered, preferably topically, to a donor organ to cause in situ expression of the soluble TGF-β antagonist in cells of the organ transfected with the vector. Such in situ expression inhibits the activity of TGF-β and curbs TGF-β-mediated fibrogenesis. Any suitable vector may be used. Preferred vectors include adenovirus, lenti virus, Epstein Barr virus (EBV), adeno associated virus (AAV), and retroviral vectors that have been developed for the purpose of gene transfer. See, e.g., Souza and Armentano, 1999, Biotechniques, 26:502-508. An adenoviral vector suitable for use in inhibiting TGF-β-mediated fibrogenesis is illustrated in the examples below. Other, non-vector methods of gene transfer may also be used, for example, lipid/DNA complexes, protein/DNA conjugates, naked DNA transfer methods, and the like.

Additional suitable TGF-β antagonists developed for delivery via adenoviral gene transfer include, but are not limited to: a chimeric cDNA encoding an extracellular domain of the TGF-β type II Receptor fused to the Ig Fc domain (Isaka et al., 1999, Kidney Int., 55:465-475), adenovirus gene transfer vector of a dominant-negative mutant of TGF-β type II Receptor (Zhao et al, 1998, Mech. Dev., 72:89-100.), and an adenovirus gene transfer vector for decorin, a TGF-β binding proteoglycan (Zhao et al., 1999, Am. J. Physiol., 277:L412-L422. Adenoviral-mediated gene transfer is very high efficiency compared to other gene delivering modalities. However, in vivo gene transfer using adenoviral vectors as a therapeutic modality has been limited by the host immune response that induces inflammation, limits the amount and duration of transgene expression, and prevents effective re-transfection. For use in the present invention, however, wherein all transplant patients the host immune response is generally suppressed, transplantation immunosuppression attenuates the post-transfection host immune response to adenoviral-mediated gene transfection and thereby increases and prolongs transgene expression. The host immunosuppression also makes effective re-transfection with adenoviral vectors possible. Thus, clinical application of gene therapy in the setting of transplantation is a preferred mode for delivery of TGF-β antagonists.

Suitable TGF-β antagonists for use in the present invention will also include functional mutants, variants, derivatives and analogues of the aforementioned TGF-β antagonists, so long as their ability to inhibit TGF-β amount or activity is retained. As used herein, “mutants, variants, derivatives and analogues” refer to molecules with similar shape or structure to the parent compound and that retain the ability to act as TGF-β antagonists. For example, any of the TGF-β antagonists disclosed herein may be crystallized, and useful analogues may be rationally designed based on the coordinates responsible for the shape of the active site(s). Alternatively, the ordinarily skilled artisan may, without undue experimentation, modify the functional groups of a known antagonist and screen such modified molecules for increased activity, half-life, bioavailability or other desirable characteristics. Where the TGF-β antagonist is a polypeptide, fragments and modifications of the polypeptide may be produced to increase the ease of delivery, activity, half-life, etc (for example, humanized antibodies or functional antibody fragments, as discussed above). Given the level of skill in the art of synthetic and recombinant polypeptide production, such modifications may be achieved without undue experimentation. Persons skilled in the art may also design novel inhibitors based on the crystal structure and/or knowledge of the active sites of the TGF-β inhibitors described herein.

TGF-β antagonists may be administered at any time that is determined to be beneficial for blocking the fibroproliferative effects of TGF-β. Preferably administration is post-transplant. Alternatively, especially for systems of administration where the bioavailability of the TGF-β antagonist is delayed or is induced, e.g., by expression of a recombinant gene implanted in the host by transfection or other means, administration can take place before or at the time of transplant. Most preferably, administration or expression of the TGF-β antagonist is made to coincide with peak TGF-β levels following introduction of the transplant organ or tissue. For example, there is an observed rise in numbers of infiltrating TGF-β-positive lymphocytes during the first week following transplant. Boehler et al., 1997, Transplantation, 64:311-317. Data presented below confirms the increased number of TGF-β-positive infiltrating cells seen at day 7 following transplant and suggests that this could also be the peak of TGF-β, which is produced from these cells. The administration of TGF-β antagonist will preferably be timed to deliver the maximum amount of antagonist at the time of maximum TGF-β expression, thus neutralizing most effectively the subsequent development of TGF-β-mediated fibroproliferation and fibrous deposition in transplant structures, e.g., the airway lumen, blood vessels, renal tubules, etc. The timing of administration of the antagonist will most advantageously be tailored to the form of antagonist. For instance, data presented herein confirm previous observations that gene transfer methods take 24-48 hours to reach the peak of transgene expression, and therefore vector delivered on day 5-6 post-transplant is designed to cause a peak of transgene expression which coincides with the peak of the infiltrating TGF-β-positive cells, leading to the most effective inhibition of graft fibrosis induced by TGF-β.

The TGF-β antagonist may be administered in any suitable way, as outlined above, and the mode of administration also will preferably be tailored to the type or activity of the antagonist employed. For example, TGF-β antagonists which downregulate TGF-β expression may be administered locally or systemically, whereas TGF-β antagonists that operate by binding to TGF-β and blocking its binding to cellular receptors will be administered locally for the best effect (see Example 5, infra). In a preferred embodiment, where a soluble TGF-β inhibitor is used, such as the case with soluble TGF-β type III Receptor via adenovirus gene transfer exemplified herein, topical injection at the site of the transplant is effective in preventing fibroproliferation.

It will be readily apparent to those skilled in the art that other suitable modifications and adaptations of the compositions and methods of the invention described herein are obvious and may be made without departing from the scope of the invention or the embodiments disclosed herein. Having now described the present invention in detail, the same will be more clearly understood by reference to the following examples, which are included for purposes of illustration only and are not intended to be limiting of the invention.

### EXAMPLE 1

### Preparation of Adenoviral Vector for Gene Transfer of a TGFB Antagonist

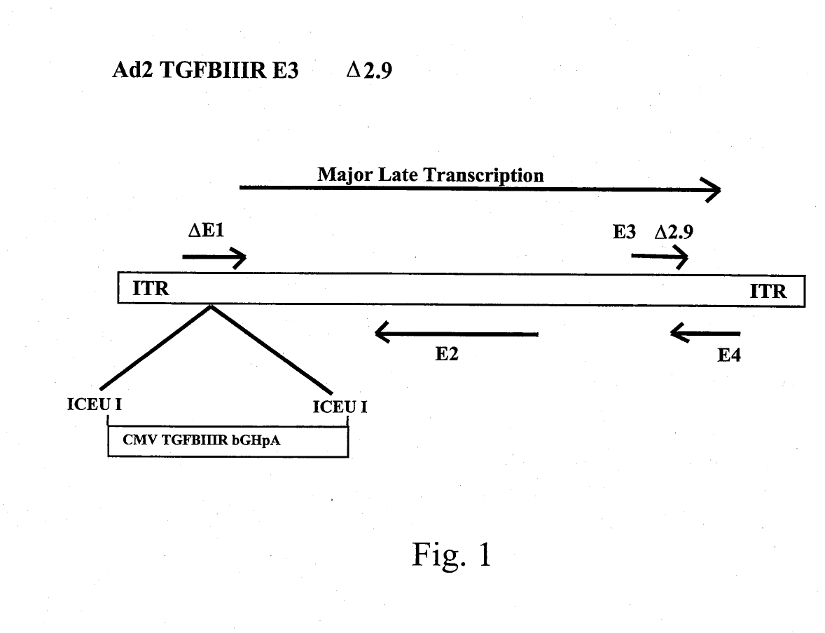

The human TGF-B type III soluble receptor was amplified from clone #7411 (Clone# 7411 was obtained from Lodish/Weinberg labs at the Whitehead Institute) (Moren et al., Biochem Biophys. Research Communications, 189:356-362(1992)) using the following primers:
TGFB-3R-II: 5′-GTAGAGCTCCACCATGACTTCCCATTATGTGATTGCCAT-3′ (SEQ ID NO:1), and
TGFBIII-3′: 5′-GTGTCTAGACTAGTCCAGACCATGGAAAATTGGTGG-3′ (SEQ ID NO:2), with Vent_{R}® DNA polymerase (New England Biolabs, Beverly, Mass.). PCR products were fractionated on a 1% agarose gel, and a 2.2 kb product was purified, digested with Ecl136 II and Xba I, and cloned into the EcoR V-Xba I site of the pAdQUICK (formerly called pAd_{vantage}) shuttle vector pSV2-ICEU I (Genzyme Corp., Cambridge, Mass.). Recombinant adenovirus was generated in a multi-step manner as illustrated in FIG. 1 of Souza et.al., 1999, Biotechniques, 26:502-8. This quick cloning system involves three steps, 1) the transgene is cloned into the shuttle vector pSV2-ICEU I, 2) it is then subcloned from pSV2-Ceu I by digestion into the I-Ceu I site of the viral vector pAdQUICK, and then 3) the pAdQUICK-based recombinant adenoviral vector is cleaved with SnaBI to expose the inverted terminal repeats. The transgene expression was confirmed by testing supernatants of complementing 293 cells (primary human embryonal kidney cells, transformed with adenovirus type 5 (Ad5), ATCC CRL-1573, Rockville, Md.) infected with recombinant adenovirus Ad2TGFBIIIR, using Western blotting, probed with goat anti-human TGFBIIIR antibody (R&D Systems, Minneapolis, Minn.).

### EXAMPLE 2

### Rat Lung Allograft Model

Male Brown-Norway and Lewis rats (approx. 200-300 g) were purchased from Harlan Sprague Dawley Inc. (Indianapolis, Ind.) and Charles River Canada, Inc. (St. Constant, Quebec, Canada), respectively. Animal care was provided according to NIH guidelines and approved by the Toronto General Hospital Research Institute Animal Care Committee.

Heterotopic tracheal transplantation was carried out as previously described (Boehler et al., 1997, Transplantion, 64:311-7; Boehler et al., 1998, Hum. Gene Ther., 9:541-51; Suga et al., 2000, Am. J. Respir. Crit. Care Med., 162:1940-1948). Briefly, entire trachea of Brown-Norway rat was excised, divided into two equal sized segments, arid then placed into a subcutaneous pouch made in the back of the recipient (Lewis rats). Grafts were removed 2, 7, 14 and 21 days after transplantation, and the middle third of the tracheal segment was fixed with 10% buffered formalin for histology and immunohistochemistry studies.

For histological staining, the graft specimens were processed as described in Suga et al., 2000, Am. J. Respir. Crit. Care Med., 162:1940-1948. Briefly, frozen specimens were embedded in O.C.T. compound (Sakura Finetek U.S.A., Inc.; Torrence, Calif.), cut into 5-μm sections, placed on poly-L-lysine-coated slides, air-dried and fixed with acetone for 15 minutes. After blocking with Protein Block Serum-Free solution (DAKO Diagnostic Canada, Inc., Mississauga, Ontario, CA), the sections were incubated with diluted polyclonal rabbit anti-TGF-β IgG (Santa Cruz Biotechnology, Santa Cruz, Calif.) at 1:100 for 30 minutes. The secondary antibody and alkaline phosphatase conjugation steps and color reaction were performed according to the manufacturer's instructions of LSAB 2 Alkaline Phosphatase Kit for Rat Specimens (DAKO Diagnostic Canada). The color reaction was developed with the addition of Fast Red Substrate System (DAKO Diagnostic Canada) including an endogenous alkaline phosphate activity blocker (Levamisole; DAKO Diagnostic Canada). Slides were counterstained with hematoxylin. Negative controls were incubated with PBS containing 0.1% bovine serum albumin without the primary antibody, or with isotype specific rabbit IgG.

For morphometry, the formalin-preserved middle portion of the tracheal segment was cut into 4-μm sections for hematoxylin and eosin staining. Computerized morphometry as described by Reichenspurner et al., 1997, Transplantation, 64:373-383) was performed by taking photoimages of the section under the microscope with an attached video camera. The images were transferred to a C-imaging 1280 morphometric system (Compix, Inc., Cranberry Township, Pa.). The luminal circumference, the epithelial lining, the margin of patent lumen, and the margin within the cartilage were traced with manual drawing and each length or area was calculated by the computer system. The percentage epithelial loss was expressed as (1−the length of epithelial lining÷the length of luminal circumference)×100%; the percentage luminal obliteration was expressed as (1−the area of patent lumen÷the area of inside cartilage)×100%. Data are expressed as mean values±standard deviation of the means. Statistical analysis was performed using SigmaStat v1.0 statistical software (Jandel Scientific; San Rafael, Calif.).

### EXAMPLE 3

### TGF-β Expression at Various Time Points after Transplantation

The fibrous airway obliteration that develops in lung allografts follows a triphasic time course: an initial ischemic phase, followed by a marked cellular infiltrate phase with complete epithelial loss, and finally a fibrous obliterative phase of the allograft airway lumen. See, Boehler et al., 1997, Transplantation, 64:311-317. The infiltrating cells are primarily lymphocytes and macrophages, especially CD4⁺ mononuclear cells.

The expression and distribution of TGF-β protein in allografted tracheal tissue was examined by immunohistochemistry staining at these three phases. Histological sections removed at different time points and examined. The results showed that the number of infiltrating mononuclear cells increased from Day 2 to Day 7, and these cells stained strongly with anti-TGF-β antibody. At Day 14, the airway lumen was filled with fibrotic tissue, and few TGF-β-positive cells could be found. At Day 21, no TGF-β-positive staining cells were found, but the fibrotic tissue was still positively stained.

### EXAMPLE 4

### TGF-β Antagonist Gene Transfer at Various Time Points

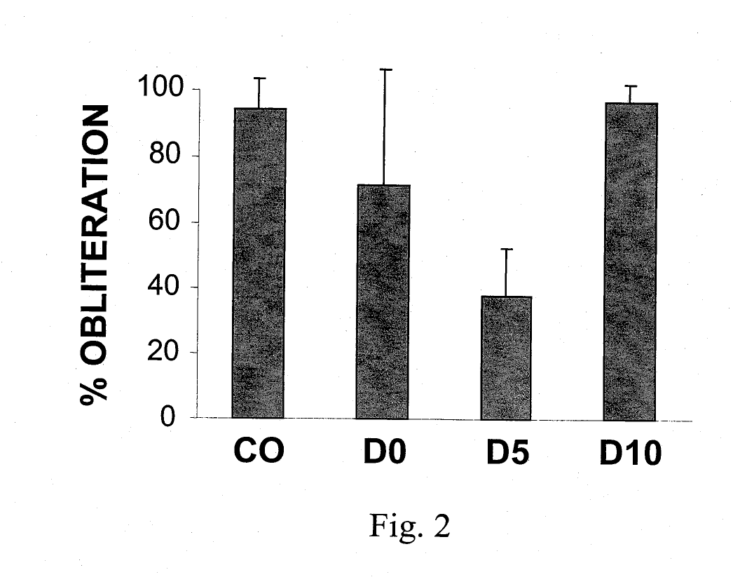

To test the effect of adenoviral mediated gene transfer of soluble TGFBIIIR on fibrous airway obliteration, recombinant adenovirus (5 x 109 particles) including the soluble TGFBIIIR gene was administered by topical injection (injection at the allograft site in recipient Lewis rats) at three different time points, i.e., day of transplant (D0), five days after transplant (D5), and ten days after transplant (D10). The results are illustrated in FIG. 2, which shows that airway obliteration was inhibited somewhat in the D0 group and to a greater extent in the D5 group, as compared with untreated control (CO).

### EXAMPLE 5

### Inhibition and Route of Administration

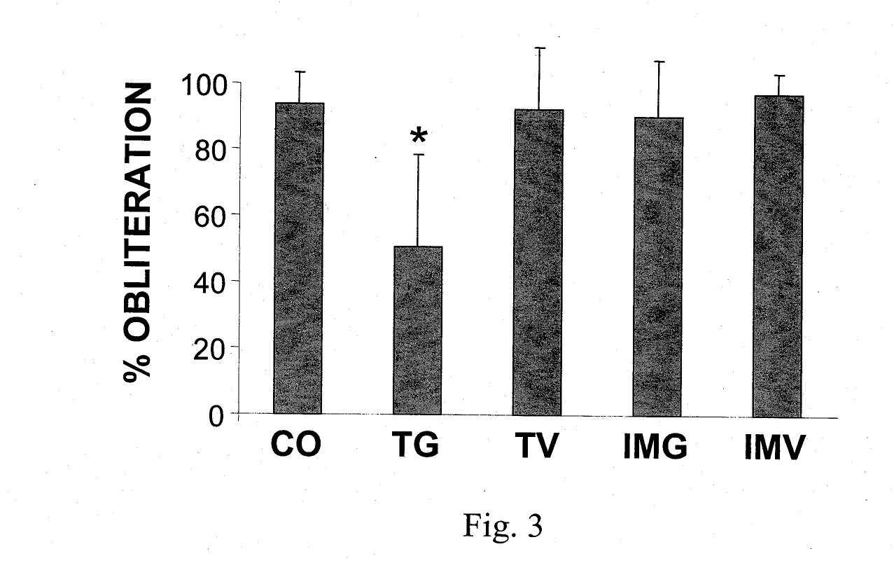

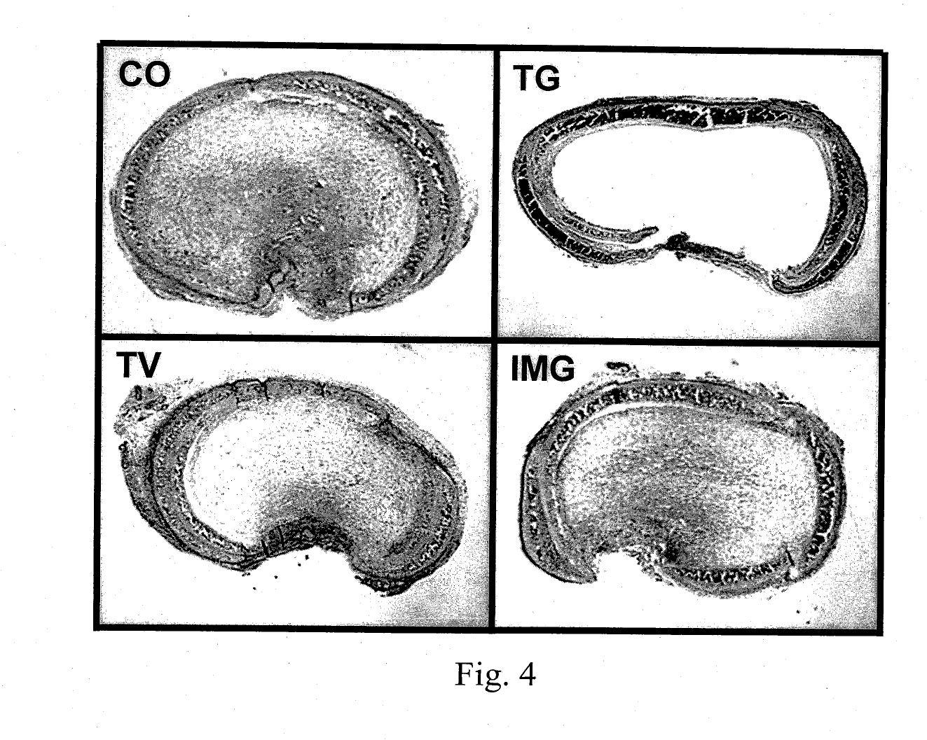

Intramuscular injection of adenoviral mediated soluble TGFBIIIR gene transfer and topical injection were tested together. Adenoviral vector containing soluble TGFBIIIR gene was injected at Day 5 after allograft transplantation either topically (at the site of tracheal transplantation) or intramuscularly (TG or IMG). The recombinant adenovirus containing an empty vector was used as a negative control (TV—empty vector, topical injection; IMV—empty vector, intramuscular injection). An untreated control group (CO) was also used. The results are illustrated in FIG. 3. Topical gene transfection of soluble TGFBIIIR (group TG) preserved lumenal patency through Day 21, while all other groups showed almost complete fibrous lumenal obliteration (FIGS. 3 and 4). On examination of tracheal structure, although topical gene transfer prevented fibrous obliteration in the airway lumen, loss of the entire epithelial lining (which was the same in all groups), was not prevented by the gene transfer. In addition, minor degrees of fibroproliferation were observed in the subepithelial space, replacing the normal architecture in that location.

The topical effect of soluble TGFBIIIR may be particularly advantageous for clinical purposes. After lung transplantation, this protein or its gene (or a similar TGF-β inhibitor) can be delivered locally through the trachea, to prevent chronic rejection from fibrous airway obliteration, while minimizing its impact systemically. Thus, the amount of the TGF-β antagonist required in a local administration will be less than with systemically acting agents, and the potential for systemic side effects will be reduced.

The foregoing results indicate that a direct inhibitor of TGF-β binding, e.g., adenoviral mediated topical gene transfer of soluble TGFBIIIR, significantly inhibits the development of allograft-induced fibrous airway obliteration in a rat tracheal transplant model of bronchiolitis obliterans. The results highlight the connection between clinically observed overexpression of TGF-β and the development of airway fibrosis that contributes to the chronic dysfunction of the grafted lung after transplantation. This provides the first direct evidence that the therapeutic approaches designed to block the activity of TGF-β are useful to inhibit chronic graft fibrosis. The strong anti-TGF-β staining of infiltrating mononuclear cells suggest that these cells could be one of the major sources of TGF-β in allograft settings.

The present invention refers to standard laboratory and scientific techniques well known in the fields of molecular biology and medicine. These techniques include, but are not limited to, techniques described in the following publications:
Ausubel, F. M. et al. eds., Short Protocols In Molecular Biology (4^{th} Ed. 1999) John Wiley & Sons, NY. (ISBN 0-471-32938-X).
Old, R. W. & S. B. Primrose, Principles of Gene Manipulation: An Introduction To Genetic Engineering (3d Ed. 1985) Blackwell Scientific Publications, Boston. Studies in Microbiology; V.2:409 pp. (ISBN 0-632-01318-4).
Sambrook, J. et al. eds., Molecular Cloning: A Laboratory Manual (2d Ed. 1989) Cold Spring Harbor Laboratory Press, NY. Vols. 1-3. (ISBN 0-87969-309-6).
Winnacker, E. L. From Genes To Clones: Introduction To Gene Technology (1987) VCH Publishers, NY (translated by Horst Ibelgaufts). 634 pp. (ISBN 0-89573-614-4).

Each of the publications mentioned hereinabove is incorporated by reference in its entirety.

We claim:

1. A method for inhibiting chronic rejection of a transplant comprising administering to a transplant recipient an amount of a TGF-β antagonist effective to reduce TGF-β-mediated fibrosis in the transplant.

2. The method of claim 1, wherein said chronic rejection is characterized by infiltration into the transplant of TGF-β-positive cells of the recipient.

3. The method of claim 1, wherein said transplant is a lung.

4. The method of claim 1, wherein said transplant is a kidney.

5. The method of claim 1, wherein said transplant is a heart.

6. The method of claim 1, wherein said transplant is a liver or a portion of a liver.

7. The method of claim 1, wherein said transplant is a valve of a heart.

8. The method of claim 1, wherein said transplant is a section of a blood vessel.

9. The method of claim 1, wherein said transplant is an allotransplant.

10. The method of claim 1, wherein said transplant is a xenotransplant.

11. The method of claim 1, wherein said recipient is a human.

12. The method of claim 1, wherein said TGF-β antagonist is selected from the group consisting of: an antibody directed against one or more isoforms of TGF-β; a TGF-β receptor; an antibody directed against one or more TGF-β receptors; latency associated peptide; large latent TGF-β a TGF-β inhibiting proteoglycan; somatostatin; mannose-6-phosphate; mannose-1-phosphate; prolactin; insulin-like growth factor II; IP-10; an arg-gly-asp containing peptide; a plant, fungal, or bacterial extract,; an antisense oligonucleotide; and a protein involved in TGF-β signaling.

13. The method of claim 12, wherein said TGF-β inhibiting proteoglycan is selected from the group consisting of: fetuin; decorin; biglycan; fibromodulin; lumican; and endoglin.

14. The method of claim 12, wherein said protein involved in TGF-β signaling is selected from the group consisting of: SMADs; MADs; Ski; and Sno.

15. The method of claim 12, wherein said antibody directed against one or more isoforms of TGF-β is a human or humanized form of monoclonal antibody 1D11.16.

16. The method of claim 12, wherein said antagonist is administered via gene transfer vector capable of expression of a gene encoding said antagonist in said recipient.

17. The method of claim 16, wherein said gene transfer vector is a recombinant adenovirus.

18. The method of claim 17, wherein said gene encodes a TGF-β receptor or a fragment thereof capable of binding to TGF-β.

19. The method of claim 18, wherein said TGF-β receptor is TGF-β type III Receptor.

20. A method for inhibiting loss of transplant function in a recipient of such transplant comprising administering to said individual a pharmaceutically effective amount of a TGF-β antagonist.

21. The method of claim 20, wherein said loss of transplant function is characterized by infiltration into the transplant of TGF-β-positive cells of the recipient.

22. The method of claim 20, wherein said transplant is a lung.

23. The method of claim 20, wherein said transplant is a kidney.

24. The method of claim 20, wherein said transplant is a heart.

25. The method of claim 20, wherein said transplant is a liver or a portion of a liver.

26. The method of claim 20, wherein said transplant is a valve of a heart.

27. The method of claim 20, wherein said transplant is a section of a blood vessel.

28. The method of claim 20, wherein said transplant is an allotransplant.

29. The method of claim 20, wherein said transplant is a xenotransplant.

30. The method of claim 20, wherein said recipient is a human.

31. The method of claim 20, wherein said TGF-β antagonist is selected from the group consisting of: an antibody directed against one or more isoforms of TGF-β; a TGF-β receptor; an antibody directed against one or more TGF-β receptors; latency associated peptide; large latent TGF-β a TGF-β inhibiting proteoglycan; somatostatin; mannose-6-phosphate; mannose-1-phosphate; prolactin; insulin-like growth factor II; IP-10; an arg-gly-asp containing peptide; a plant, fungal, or bacterial extract,; an antisense oligonucleotide; and a protein involved in TGF-β signaling.

32. The method of claim 31, wherein said TGF-β inhibiting proteoglycan is selected from the group consisting of: fetuin; decorin; biglycan; fibromodulin; lumican; and endoglin.

33. The method of claim 31, wherein said protein involved in TGF-β signaling is selected from the group consisting of: SMADs; MADs; Ski; and Sno.

34. The method of claim 31, wherein said antibody directed against one or more isoforms of TGF-β is a human or humanized form of monoclonal antibody 1D11.16.

35. The method of claim 31, wherein said antagonist is administered via gene transfer vector capable of expression of a gene encoding said antagonist in said recipient.

36. The method of claim 35, wherein said gene transfer vector is a recombinant adenovirus.

37. The method of claim 36, wherein said gene encodes a TGF-β receptor or a fragment thereof capable of binding to TGF-β.

38. The method of claim 37, wherein said TGF-β receptor is TGF-β type III Receptor.
